(19) United States
(12) Patent Application Publication
(10) Pub. No.: US 2005/0070577 A1
(43) Pub. Date: Mar. 31, 2005

(54) Compositions containing a serotonin selective reuptake inhibitor and a 5-HT2A receptor antagonist

(75) Inventors: Gerard J. Marek, East Lyme, CT (US); Robert Mark Berman, New Haven, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/870,127

(22) Filed: Jun. 17, 2004

Related U.S. Application Data
(60) Provisional application No. 60/484,950, filed on Jul. 3, 2003.

(51) Int. Cl. A61K 31/137; A61K 31/445

(52) U.S. Cl. 514/317; 514/649

(57) ABSTRACT

Novel compositions comprising a selective serotonin re-uptake inhibitor (SSRI), particularly sertraline, and a selective inhibitor for the binding of serotonin at the 5HT_{2A} site. The invention relates to the use of such compositions for treating or preventing mood disorders including depression and anxiety disorders.

### FIELD OF THE INVENTION

This invention pertains to novel compositions comprising a selective serotonin re-uptake inhibitor (SSRI) and a selective inhibitor for the binding of serotonin at the 5HT_{2A} site. In a particular aspect, this invention relates to novel compositions containing the serotonin selective re-uptake inhibitor (1 S-cis)-4-(3,4-dichlorophenyl)-1.2.3.4-tetrahydro-N-methyl-1-naphthaleneamine (hereinafter sertraline). This invention further relates to the use of such compositions for treating or preventing mood disorders including depression and anxiety disorders.

### BACKGROUND OF THE INVENTION

Serotonin (5-hydroxytryptamine, 5-HT) plays a significant role in the functioning of the central nervous system (CNS) of the mammalian body. Biochemical evidence indicates that large concentrations of 5-HT exist in the brain and spinal cord. Not surprisingly it has been found that serotonin is involved in a number of diseases related to a wide range of 5-HT binding or receptor sites. These include the areas of depression, schizophrenia, sleeping, eating, pain and blood pressure control.

Receptor-specific antagonists for 5-HT are of great interest for the treatment and control of CNS disorders including anxiety, depression, hypertension, compulsive disorders, schizophrenia, autism and neurodegenerative disorders such as Alzheimer's disease and Parkinsonism.

For example the drug sertraline has found extensive use in the treatment of CNS disorders in mammals as disclosed in e.g. U.S. Pat. Nos. 4,536,518, 4,962,128, 4,045,488 and 5,597,826 which are incorporated herein by reference therein in their entirety.

The discovery of multiple populations of binding sites for 5-HT with structurally distinct cell surface properties has led to the classification of seven major sub-types, 5-HT₁, 5-HT₂, 5-HT₃-5HT₄-5HT₅ 5-HT₆ and 5-HT₇. The potential exists for the development of therapeutic agents which are selective antagonists at the various subtype and which may exhibit selective, enhanced activity with fewer side effects. Serotonin re-uptake inhibitors including sertraline are effective serotonin antagonists at the 5HT₁ site.

The subtype 5-HT₂ is of special interest in the CNS field since it is a receptor found in brain cells and may have a role in various mental disorders such as schizophrenia hallucinations, depression, and anxiety.

Blocking of serotonin receptors at the 5-HT₂ site has led to a number of useful therapeutic effects in the CNS field as disclosed in e.g. U.S. Pat. No. 5,169,096, WO 95/24194 and WO 91/18602. The foregoing patent and patent applications are incorporated herein in their entirety.

### SUMMARY OF THE INVENTION

In one aspect, the invention relates to a pharmaceutical composition for treating depression, anxiety disorders, autism, dyskinesia, panic disorder, bipolar disorder, major depression episode of the mild, moderate or severe type, generalized anxiety disorder, social phobia, disthymic disorder, and organic impairments including dementia, mental handicap and Alzheimer's disease in a mammal, the pharmaceutical composition comprising;
(a) a 5HT_{2A} selective receptor antagonist or a pharmaceutically acceptable salt thereof;
(b) an SSRI or a pharmaceutically acceptable salt thereof; and
(c) a pharmaceutically acceptable carrier;

wherein the antagonist in (a) and the inhibitor in (b) are present in amounts that render said pharmaceutical composition effective in treating or preventing the above stated condition.

The term selective serotonin re-uptake inhibitor, SSRI, as used herein denotes a species which inhibits the uptake or re-uptake of serotonin (also known as 5-HT and 5-hydroxytryptamine) into nerve cells.

Suitably the SSRI is a selective serotonin re-uptake inhibitor such as sertraline, paroxetine, fluvoxamine, fluoxetine, femoxetine, citalopram, clomipramine, cianopramine, litoxetine, cericlamine and seproxetine.

In a preferred embodiment, the SSRI is sertraline or a pharmaceutically acceptable salt thereof.

In a specific embodiment of the invention the suitable 5HT_{2A} receptor antagonists include compounds of the formula 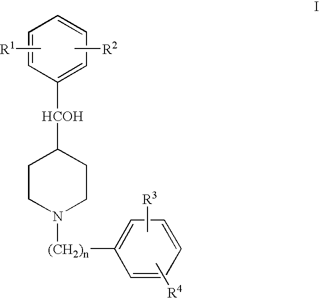
wherein n is 2,3, or 4;

and R¹, R², R³ and R⁴ are each independently hydrogen, halogen, trifluoromethyl, C₁₋₆ alkyl, C₁₋₆ alkoxy, hydroxy or amino; the optical isomers thereof and the pharmaceutically acceptable salts thereof. This compound is a selective inhibitor of the binding of serotonin at the 5HT_{2A} receptor site.

In another aspect of the invention the suitable 5HT_{2A} receptor antagonists include compounds of the formula 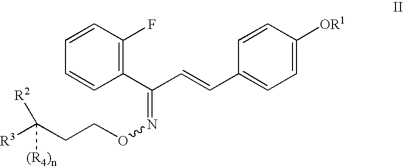
wherein

R¹ is H, glucuronide or sulfate;

R² and R³ are independently H or methyl;

R⁴ is 0, or glucuronide, and

n is 0 or 1, provided that when R is H, n is 1.

As used herein, glucuronide, also known as glucuranoside, is intended to refer to a compound in which glucuronic acid, combined as sugar (hexose), not as an acid, is linked by a glycosidic bond to a group e.g., a hydroxyl or carbonyl group, or another compound.

Most preferably, the compound of formula I is a compound of formula 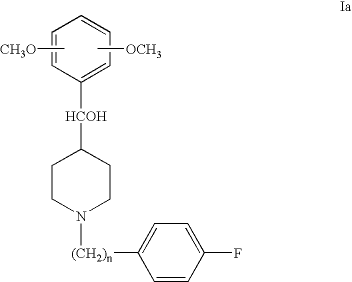
wherein n is 2 or 3;

and the most preferred compound of Formula II is a compound of formula 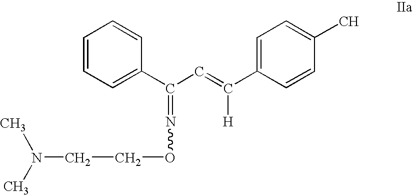
wherein the geometry with respect to the carbon double bond in trans and the geometry with respect to the carbon- nitrogen double bond is anti.

In a preferred composition of the present invention the dosage of 5HT_{2A} receptor antagonist or a pharmaceutically acceptable salt thereof is about 2 mg to about 10 mg and the amount of serotonin re-uptake inhibitor or a pharmaceutically acceptable salt thereof is about 25 mg to about 200 mg. In a more preferred composition, the dosage of 5HT_{2A} receptor or a pharmaceutically acceptable salt thereof is from about 4 mg to about 6 mg and the dosage of serotonin re-uptake inhibitor or a pharmaceutically acceptable salt thereof is about 50 mg to about 100 mg.

Preferably the 5HT_{2A} receptor antagonist is selected from:
alpha-(2,3-dimethoxyphenyl)-1-(2-phenylethyl)-4-piperidinemethanol;
alpha-(2,3-dimethoxyphenyl)-1-[2-(4-fluoro)phenylethyl]-4-piperidinemethanol;
alpha-(2,3-dimethoxyphenyl)-1-[3-(4-fluoro)phenylethyl]-4-piperidinemethanol;
alpha-phenyl-1-(2-phenylethyl)-4-piperidinemethanol;
alpha-(3,5-dimethoxyphenyl)-1-[2-(4-fluoro)phenylethyl]-4-piperidinemethanol;
alpha-(3,5-dimethylphenyl)-1-[2-(4-fluoro)phenylethyl]-4-piperidinemethanol;
alpha-phenyl-1-(3-phenylpropyl)-4-piperidinemethanol; and
alpha-(3-chlorophenyl)-(3-phenylpropyl)-4-piperidinemethanol.
   In a more preferred embodiment, the 5HT_{2A} inhibitor is selected from:
alpha-(2,3-d imethoxyphenyl)-1-[2-(4-fluoro)phenylethyl]-4-piperidinemethanol and
trans-1-N,N-dimethylaminoethoxyimino-1-(2-fluorophenyl)-3-(4-hydroxyphenyl)-2-propene.

In another aspect, the invention relates to a method for treating or preventing disorders arising from deficient or excessive serotonergic neurotransmission in a mammal, preferably a human, comprising administering to said mammal requiring such treatment or prevention
(a) a 5HT_{2A} selective receptor antagonist or a pharmaceutically acceptable salt thereof;
(b) an SSRI or a pharmaceutically acceptable salt thereof; and
(c) a pharmaceutically acceptable carrier;

wherein the antagonist in (a) and the inhibitor in (b) are present in amounts that render said pharmaceutical composition effective in treating or preventing such condition.

In a preferred embodiment, the serotonin re-uptake inhibitor is sertraline or a pharmaceutically acceptable salt thereof.

In a specific embodiment of the invention the suitable 5HT_{2A} receptor antagonists include compounds of the formula 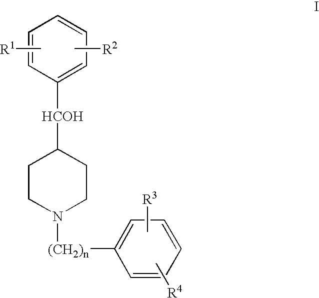
the optical isomers thereof and the pharmaceutically acceptable salts thereof wherein n is 2, 3, or4;

and R¹, R², R³ and R⁴ are each independently hydrogen, halogen, trifluoromethyl, C₁₋₆ alkyl, C₁₋₆ alkoxy, hydroxy or amino.

In another aspect of the invention the suitable 5HT_{2A} receptor antagonists comprise compounds of the formula 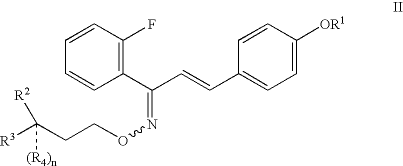
wherein

R¹ is H, glucuronide or sulfate;

R² and R³ are independently H or methyl;

R⁴ is 0, or glucuronide, and

n is 0 or 1, provided that when R is H, n is 1.

Preferably the 5HT_{2A} selective receptor antagonist is selected from:
alpha-(2,3-dimethoxyphenyl)-1-(2-phenylethyl)-4-piperidinemethanol;
alpha-(2,3-dimethoxyphenyl)-1-[2-(4-fluoro)phenylethyl]-4-piperidinemethanol;
alpha-(2,3-dimethoxyphenyl)-1-[3-(4-fluoro)phenylethyl]-4-piperidinemethanol;
alpha-phenyl-1-(2-phenylethyl)-4-piperidinemethanol;
alpha-(3,5-dimethoxyphenyl)-1-[2-(4-fluoro)phenylethyl]-4-piperidinemethanol;
alpha-(3,5-dimethylphenyl)-1-[2-(4-fluoro)phenylethyl]-4-piperidinemethanol;
alpha-phenyl-1-(3-phenylpropyl)-4-piperidinemethanol; and
alpha-(3-chlorophenyl)-(3-phenylpropyl)-4-piperidinemethanol.

In a more preferred embodiment, the 5HT_{2A} inhibitor is selected from:
alpha-(2-3-dimethoxyphenyl)-1-[2-(4-fluorophenylethyl]-4-piperidinemethanol and
trans-1-N,N-dimethylaminoethoxyimino-1-(2-fluorophenyl)-3-(4-hydroxyphenyl)-2-propene.

### DETAILED DESCRIPTION OF THE INVENTION

The 5HT_{2A} inhibitor compounds of Formula I can be prepared by the synthetic method which is described and referred to in U.S. Pat. No. 5,169,096 and illustrated in Scheme I below. 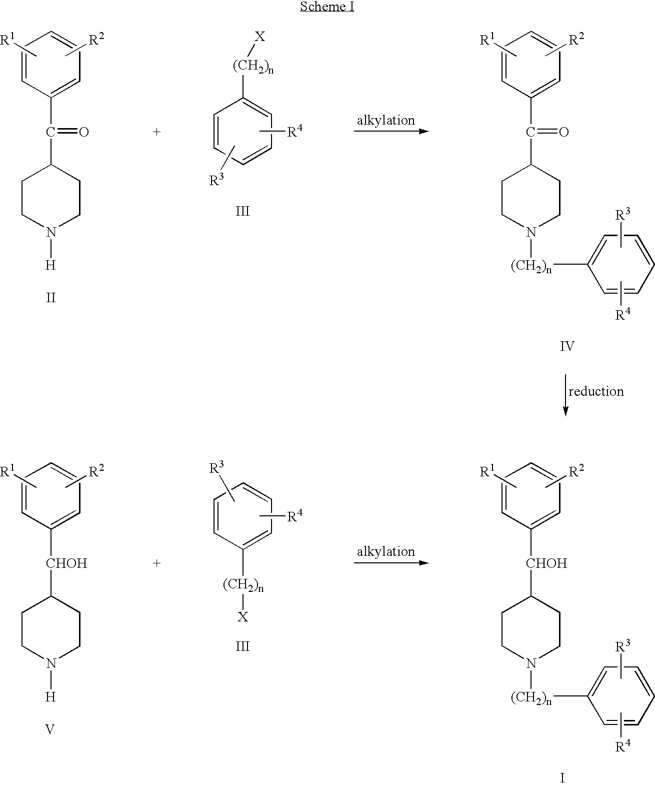
wherein n is 2, 3, or 4; and R¹, R², R³, R⁴ are each independently hydrogen, halogen, trifluoromethyl, C₁₋₆alkyl, C₁₋₆alkoxy, hydroxy, or amino.

In a preferred embodiment, the compound of Formula I is a compound wherein R¹ and R² are each methoxy, R³ is hydrogen, R⁴ is fluorine and n is 2 or 3.

The compounds of Formula II are prepared by the synthetic method which is described and referred to in U.S. Pat. No. 5,166,416 incorporated herein as a reference thereto in its entirety.

The synthesis of compounds of Formula II wherein R⁴ and n are 0, is illustrated in Scheme 2 below. 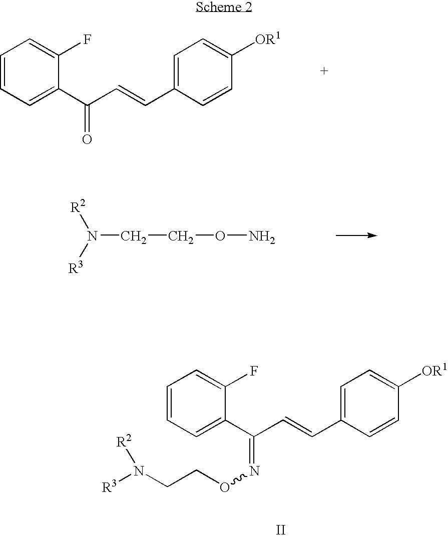
wherein R¹, R², and R³ have the same meaning as above.

The compounds of Formula II are propenone oxide ether which are configured in the trans geometry with respect to the carbon-carbon double bond.

With respect to the carbon-nitrogen double bond of the oxygen substituted oxime, the compounds of Formula II exist as a mixture of syn and anti isomers in various proportions.

In a preferred embodiment, the compound of Formula II is a compound having the formula 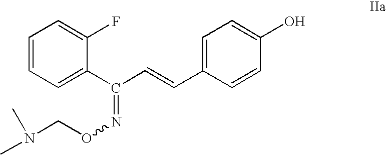
wherein the geometry with respect to the carbon-carbon double bond is trans and the geometry with respect to the carbon-nitrogen double bond is anti.

The 5-HT_{2A} receptor antagonist may be administered simultaneously, separately or sequentially relative to the SRI.

The compounds of the invention are generally administered as pharmaceutical compositions in which the active agent is mixed with a pharmaceutical excipient or carrier. The active compound or agent may be formulated for oral, buccal, intramuscular, parenteral (e.g. intravenous, intramuscular or subcutaneous) or rectal administration or in a form suitable for administration by inhalation or insufflation.

Suitable forms of oral administration include tablets, capsules, powders, granules and oral solutions or suspensions, sublingual and buccal forms of administration.

When a solid composition is prepared in tablet form, the main excipient is mixed with a pharmaceutical excipient such as gelatin, starch, lactose, magnesium stearate, talc or gem arabic. Tablets may be coated with a suitable substance like sugar so that a given quantity of the active compound is released over a prolonged period of time.

Liquid preparations for oral administration may be in the form of a solution, syrup, or suspension. Such liquids may be prepared by conventional methods using pharmaceutically acceptable ingredients such as suspending agents (e.g. sorbitol syrup); emulsifying agents (e.g. lecithin); non-aqueous vehicles (e.g. ethyl alcohol); and preservatives (e.g. sorbic acid).

Formulations for parenteral administration by injection or a infusion may be presented in unit dosage form e.g. in ampules in the form of solutions or emulsions in oily or aqueous vehicles.

The compositions may also be formulated in rectal formulations such as suppositories or retention enemas.

For intranasal or inhalation administration, the compounds are delivered in the form of a solution or suspension from a pump spray or a container pressurized with suitable propellant.

In connection with the use of the compositions of the present invention, compounds of formula I or II with an SSRI, preferably sertraline, it is to be noted that these compounds may be administered either alone or in combination with a pharmaceutically acceptable carrier. Such administration may be carried out in single or multiple doses. More particularly the composition may be combined with various pharmaceutically acceptable inert carriers in the form of tablets, capsules, lozenges, hard candies, powders, syrup, aqueous suspension, injectable solutions, elixirs, syrups, and the like.

The affinity of a compound for the 5-HT_{2A} sites is determined by the procedure of R. A. Lyon et al., Mo. Pharmcol., 1987, 31, 194-199. Biological assays for the effectiveness of the compounds of Formula I and II are describes in U.S. Pat. Nos. 5,169,096 and 5,844,000 both of which are incorporated herein by reference in their entirety.

Sertraline is a highly selective and potent inhibitor of synaptosomal serotonin reuptake with an IC₅₀ value of 0.058×10⁻⁶M. The neurochemical, behavioral and pharmacological characteristics of sertraline is described by Koe, Kenneth B. et al, Journal of Pharacology and Experimental Therapeutics., 226, 686-700 (1983) and Cusack. B., et al, Psychopharmacology, 114, 559-565 (1994).

1. A pharmaceutical composition for treating depression, anxiety disorders, autism, dyskinesia, panic disorder, bipolar disorder, major depression episodes of the mild, moderate or severe type, generalized anxiety disorder, social phobia, disthymic disorder and organic impairments including dementia, mental handicap and Alzheimer's disease in a mammal comprising:
(a) a 5HT_{2A} selective receptor antagonist or a pharmaceutically acceptable salt thereof;
(b) a selective serotonin re-uptake inhibitor (SSRI) or a pharmaceutically acceptable salt thereof; and
(c) a pharmaceutically acceptable carrier;
wherein the antagonist in (a) and the inhibitor in (b) are present in amounts that render said pharmaceutical composition effective in treating or preventing such condition.

2. The pharmaceutical composition according to claim 1 wherein said SSRI is selected from sertraline, paroxetine fluvoxamine, fluoxetine, femoxetine, citalopram, clomipramine, cianopramine, litoxetine, cericlamine and seproxetine.

3. A pharmaceutical composition according to claim 2, where said SSRI is sertraline or a pharmaceutically acceptable salt or polymorph thereof.

4. A pharmaceutical composition according to claim 1 wherein said 5HT_{2A} selective receptor is a compound having the formula 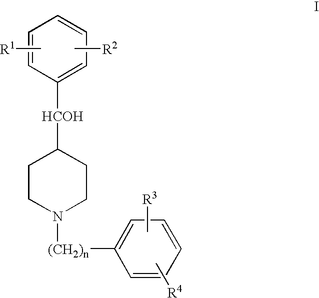
the optical isomers thereof and the pharmaceutically acceptable salts thereof wherein n is 2, 3, or 4; and R¹, R², R³, R⁴ are each independently hydrogen, halogen, trifluoromethyl, C₁₋₆ alkyl, C₁₋₆ alkoxy, hydroxy or amino.

5. A pharmaceutical composition according to claim 1 wherein said 5HT_{2A} selective receptor antagonist is a compound having the formula. 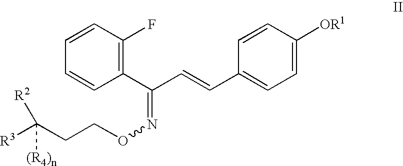
wherein
R¹ is H, glucuronide or sulfate;
R² and R³ are independently H or methyl;
R⁴ is 0, or glycuronide, and
n is 0 or 1, provided that when R is H, n is 1.

6. A pharmaceutical composition according to claim 1 wherein the amount of 5HT_{2A} receptor antagonist or pharmaceutically acceptable salt thereof in said composition is about 2 mg to about 10 mg and the amount of the serotonin re-uptake inhibitor or pharmaceutically acceptable salt thereof is about 25 mg to about 200 mg.

7. A pharmaceutical composition according to claim 6 wherein the amount of 5HT_{2A} receptor antagonist is about 4 mg to about 6 mg and the amount of the serotonin re-uptake inhibitor is about 50 mg to about 100 mg.

8. A pharmaceutical composition according to claim 4 wherein said compound of Formula I is a compound of the formula 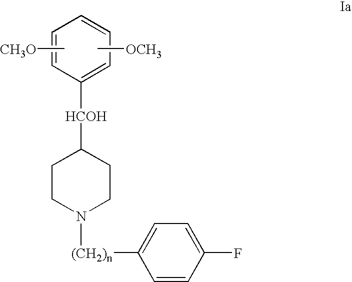
wherein n is 2 or 3.

9. A pharmaceutical composition according to claim 5 wherein said compound of Formula II is a compound of the formula 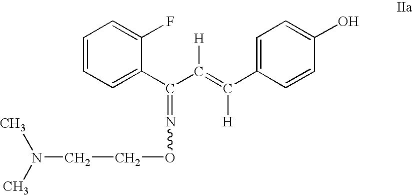
wherein the geometry with respect to the carbon-carbon double bond is trans and the geometry with respect to the carbon-nitrogen double bond is anti.

10. A pharmaceutical composition according to claim 1 wherein said 5HT_{2A} selective receptor antagonist is selected from:
alpha-(2,3-dimethoxyphenyl)-1-(2-phenylethyl)-4-piperidinemethanol;
alpha-(2,3-dimethoxyphenyl)-1-[2-(4-fluoro)phenylethyl]-4-piperidinemethanol;
alpha-(2,3-dimethoxyphenyl)-1-[3-(4-fluoro)phenylethyl]-4-piperidinemethanol;
alpha-phenyl-1-(2-phenylethyl)-4-piperidinemethanol;
alpha-(3,5-dimethoxyphenyl)-1-[2-(4-fluoro)phenylethyl]-4-piperidinemethanol;
alpha-(3,5-dimethylphenyl)-1-[2-(4-fluoro)phenylethyl]-4-piperidinemethanol;
alpha-phenyl-1-(3-phenylpropyl)-4-piperidinemethanol;
alpha-(3-chlorophenyl)-(3-phenylpropyl)-4-piperidinemethanol and
trans-1-N,N-dimethylaminoethoxyimino-1-(2-fluorophenyl)-3-(4-hydroxyphenyl)-2-propene.

11. A pharmaceutical composition according to claim 10 wherein said 5HT_{2A} selective receptor antagonist is selected from:
alpha-(3-chlorophenyl)-(3-phenylpropyl)-4-piperidinemethanol and
trans-1-N,N-dimethylaminoethoxyimino-1-(2-fluorophenyl)-3-(4-hydroxyphenyl)-2-propene.

12. A method of treating depression, anxiety disorders, autism, and organic impairments including dementia, mental handicap and Alzheimer's disease in a mammal, comprising administering to said mammal a therapeutically effective amount of a pharmaceutical composition comprising
(a) a 5HT_{2A} receptor antagonist;
(b) an SSRI or a pharmaceutically acceptable salt-thereof; and
(c) a pharmaceutically acceptable carrier;
wherein the antagonist in (a) and the inhibitor in ( b) are present in amounts that render said pharmaceutical composition effective in treating or preventing such condition.

13. A method according to claim 9 wherein said 5HT_{2A} selective receptor antagonist is a compound having the formula: 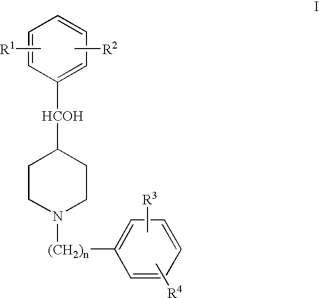
the optical isomers thereof and the pharmaceutically acceptable salts thereof
wherein n is 2, 3, or 4; and R¹, R², R³, R⁴ are each independently hydrogen, halogen, trifluoromethyl, C₁₋₆ alkyl, C₁₋₆ alkoxy, hydroxy or amino.

14. A method according to claim 12 wherein said 5HT_{2A} selective receptor antagonist is a compound having the formula 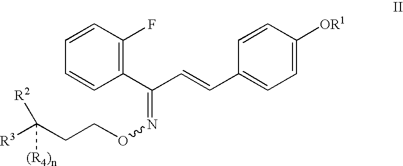
wherein
R¹ is H, glucuronide or sulfate;
R² and R³ are independently H or methyl;
R⁴ is 0 or glucuronide, and
n is 0 or 1, provided that when R is H, n is 1.

15. A method according to claim 12 wherein said SSRI is sertraline or a pharmaceutically acceptable salt or polymorph thereof.
